(19) United States
(12) Patent Application Publication
(10) Pub. No.: US 2025/0285724 A1
(43) Pub. Date: Sep. 11, 2025

(54) Novel Treatment Method for Painful Musculoskeletal Conditions

(71) Applicant: Anuli Mkparu, Philadelphia, PA (US)

(72) Inventor: Anuli Mkparu, Philadelphia, PA (US)

(21) Appl. No.: 19/071,716

(22) Filed: Mar. 5, 2025

Related U.S. Application Data
(60) Provisional application No. 63/561,729, filed on Mar. 5, 2024.

(51) Int. Cl. G16H 20/10; A61B 5/00; A61B 5/16; G16H 20/30; G16H 20/60; G16H 20/70

(52) U.S. Cl. CPC G16H 20/10; A61B 5/165; A61B 5/4824; G16H 20/30; G16H 20/60; G16H 20/70

(57) ABSTRACT

A value-based method of treating musculoskeletal pain is described, which includes care for the exacerbating coexisting physical and behavioral health concerns and the social determinants of health; all without bundling in surgery. In general, the techniques described may relate to a specific combination of treatment methodologies, including pain management, physical therapy, physical fitness and nutrition, weight management, behavioral health, substance abuse treatment, smoking cessation, reliable case management, and prompt referral for acute or severe pathological conditions.

### CROSS-REFERENCE TO RELATED APPLICATIONS

This application claims the benefit under 35 U.S.C. § 119(e) of U.S. Provisional Patent Application No. 63/561,729 titled “Novel Treatment Method for Painful Musculoskeletal Conditions” filed on 5 Mar. 2024, which disclosure is incorporated herein by reference in its entirety.

### TECHNICAL FIELD

This disclosure relates to orthopaedic care and more specifically relates to non-surgical treatment of painful musculoskeletal conditions.

### BACKGROUND

Orthopaedic surgeons are the only physicians who are trained in the management of the entire scope of all musculoskeletal conditions. There are currently about 30,000 orthopaedic surgeons in the United States. The treatment of musculoskeletal conditions has traditionally focused on three types of interventions: pharmacology (including the use of opiates/narcotics), physical therapy and surgery. A patient experiencing musculoskeletal pain would be referred (typically by a primary care provider) to an orthopaedic surgeon, who would prescribe pain medication (sometimes including opiates/narcotics) and typically recommend surgery within the first few appointments. This pathway would typically also involve post-operative pain medication (including opiates/narcotics) and post-operative physical therapy. If the surgeon does not deem the patient to have an operative problem, they are typically prescribed pain medication and recommended to get physical therapy and told there is no need to follow-up with the surgeon again. Research shows that musculoskeletal pain very often co-exists and is often exacerbated by other physical and behavioral health conditions, which themselves cannot be treated effectively by surgery. Research also shows that a patient's health is also significantly affected by social determinants of health (SDOH). In spite of this, the current reimbursement structure by insurance companies/payers incentivizes surgeons to operate more by paying so much more for surgeries rather than incentivizing them to maintain the patient's musculoskeletal health. This has led to a very siloed approach to musculoskeletal care, which provides sub-optimal patient outcomes and causes the payers and health care system to waste a lot of money.

In recent years, the healthcare industry has begun focusing more on value-based care, which includes a focus on quality of care, provider performance, and patient experience. In value-based care, physicians and other health care providers work together to manage a patient's overall health while considering the patient's personal health goals. Despite this shift, there is still no value-based method of care for orthopaedic patients that does not include surgery. In particular, no specific methodology has ever been articulated for value-based care of musculoskeletal pain, which includes care for the exacerbating coexisting physical and behavioral health concerns and the social determinants of health; all without bundling in surgery.

### SUMMARY

In some aspects, the techniques described herein relate to a value-based method of treating musculoskeletal conditions which includes care for the exacerbating coexisting physical and behavioral health concerns and the social determinants of health; all without bundling in surgery. In general, the techniques described herein may relate to a specific combination of treatment methodologies, including pain management, physical therapy, physical fitness and nutrition, weight management, behavioral health, substance abuse treatment, smoking cessation, reliable case management, and prompt referral for acute or severe pathological conditions.

The method may include conducting an initial consultation with a patient. The initial consultation may be performed by a physician, a physical therapist (PT), and a licensed clinical social worker (LCSW). During the initial consultation, the method may include determining the musculoskeletal condition of the patient. Based on the musculoskeletal condition, the method may include determining, during the initial consultation, a pain medication protocol for the patient. The method may include determining, during the initial consultation, a physical therapy protocol for the patient. A diet and fitness protocol may be determined for the patient. A weight loss medication protocol may be determined for the patient. Additionally, during the initial consultation, the method may include determining a behavioral health condition of the patient. The method may include determining, during and in connection with the initial consultation, whether the patient has opiate use disorder (OUD). The method may include determining, during the initial consultation, a case management protocol for the patient, which also addresses the SDOH. During the initial consultation, it may be determined whether the patient smokes. Additionally, the method may include determining during the initial consultation, in connection with the testing of the blood, or in connection with imaging performed on the patient, whether to refer the patient to a specialist.

In various implementations of the method, the pain medication protocol may include instructions for administering to the patient one or more of methylprednisolone, lidocaine, amitriptyline, meloxicam, acetaminophen, cyclobenzaprine, and a nonsteroidal anti-inflammatory drug (NSAID). In some implementations, the pain medication protocol does not include an opiate, quetiapine, benzodiazepine, or a stimulant. The pain medication protocol may be based on the patient indicating a desire for a pain medication. The method may include administering the pain medication protocol to the patient.

In various implementations, the physical therapy protocol may include performance of one or more exercises. The protocol may prescribe performance of the one or more exercises two times a week for six weeks, and a re-evaluation of the patient may be performed at a conclusion of the physical therapy protocol. The method may include providing the physical therapy protocol to the patient.

The method may include, in various implementations, obtaining height and weight information from the patient. Such information may be obtained in connection with the initial consultation. The method may include determining, based on the height and weight information, a body mass index (BMI) of the patient. In response to the BMI of the patient exceeding a threshold BMI, the method may include providing the diet and fitness protocol to the patient.

In various implementations, the method may include obtaining, in connection with the initial consultation, blood and urine from the patient. Testing may be performed on the blood, which testing may include one or more of a metabolic panel, a lipid panel, a thyroid panel, a diabetes panel, a nutrition panel, and a vitamin panel. The method may include performing a toxicology test on the urine to determine whether the patient has engaged in substance abuse. Various protocols may be determined based on one or more results of the blood testing and the toxicology test.

In some implementations, the method may include prescribing a weight loss medication. The weight loss medication protocol may include one or more of phentermine, orlistat, and semaglutide. The method may include administering the weight loss medication protocol to the patient.

The method may include, in various implementations, determining, based on the behavioral health condition of the patient, a behavioral health treatment protocol for the patient. The behavioral health treatment protocol may include one or more of non-facilitated self-help, guided self-help, counseling, cognitive behavioral therapy (CBT), behavioral activation (BA), physical exercise, mindfulness, meditation, interpersonal psychotherapy (IPT), and short-term psychodynamic psychotherapy (STPP). The method may include providing the behavioral health treatment protocol to the patient.

In various implementations, the method may include determining, based on the behavioral health condition of the patient, a behavioral medication protocol for the patient. The behavioral medication including one or more of a selective serotonin reuptake inhibitor (SSRI), a serotonin and norepinephrine reuptake inhibitor (SNRI), an anticonvulsant, an anxiolytic, a tricyclic antidepressant (TCA), a tetracyclic antidepressant (TeCA), a monoamine oxidase inhibitor (MAOI), a betablocker, an antihistamine, an antipsychotic, and an herbal compound, and wherein the behavioral health treatment protocol does not include benzodiazepine or an antipsychotic. The method may include administering the behavioral medication protocol to the patient.

In various implementations, the patient may indicate OUD. The method may include, in response to the patient indicating OUD, determining an OUD treatment protocol for the patient. The OUD treatment protocol may include prescribing buprenorphine and naloxone for two weeks, a follow-up appointment, and a follow-up toxicology test.

The case management protocol may, in various implementations, include monthly monitoring by a case manager of the patient's social determinants of health. The case management protocol may include monitoring reports from a prescription drug monitoring program that indicates prescriptions to the patient. The method may include providing the case management protocol to the case manager.

In various implementations, the patient may be a smoker. In response to determining the patient smokes, the method may include determining a smoking cessation protocol for the patient. The smoking cessation protocol may include one or more of a nicotine patch, varenicline, and bupropion. The method may include administering the smoking cessation protocol to the patient.

In various implementations, determining whether to refer the patient may be based on one or more of vitals of the patient, abnormal lab results, the imaging, and the patient reporting one or more of unintentional weight loss, unexplained night sweats, active vomiting, active diarrhea, current fever, complaint by the patient of objective muscle weakness, acute problems with bowel or bladder function, dizziness, chest pain, and dyspnea. In response to determining to refer the patient to a specialist or other physician, the method may include providing referral information to the patient.

### BRIEF DESCRIPTION OF THE DRAWINGS

The present description will be understood more fully when viewed in conjunction with the accompanying drawings of various examples of a method of treating a musculoskeletal condition. The description is not meant to limit the method of treating a musculoskeletal condition to the specific examples. Rather, the specific examples depicted and described are provided for explanation and understanding of the method of treating a musculoskeletal condition. Throughout the description the drawings may be referred to as drawings, figures, and/or FIGS.

FIGS. 1A-C illustrate a method of treating a musculoskeletal condition, according to an implementation.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

As used herein, “and/or” includes any and all combinations of one or more of the associated listed items.

As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, Band C; A, B, C, and D; and so forth. The bounds of an “and/or” list are defined by 2148 the complete set of combinations and permutations for the list.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein “same” means sharing all features and “similar” means sharing a substantial number of features or sharing materially important features even if a substantial number of features are not shared. As used herein “may” should be interpreted in a permissive sense and should not be interpreted in an indefinite sense. Additionally, use of “is” regarding examples, elements, and/or features should be interpreted to be definite only regarding a specific example and should not be interpreted as definite regarding every example. Furthermore, references to “the disclosure” and/or “this disclosure” refer to the entirety of the writings of this document and the entirety of the accompanying illustrations, which extends to all the writings of each subsection of this document, including the Title, Background, Brief description of the Drawings, Detailed Description, Claims, Abstract, and any other document and/or resource incorporated herein by reference.

A method of treating a musculoskeletal condition as disclosed herein will become better understood through a review of the following detailed description in conjunction with the figures. The detailed description and figures provide merely examples of the various embodiments of the method. Many variations are contemplated for different applications and design considerations; however, for the sake of brevity and clarity, all the contemplated variations may not be individually described in the following detailed description. Those skilled in the art will understand how the disclosed examples may be varied, modified, and altered and not depart in substance from the scope of the examples described herein.

As described above, a conventional method of treating a musculoskeletal condition may include prescribing a narcotic or opiate, performing surgery, and/or physical therapy. However, this ignores the research that indicates many, if not most musculoskeletal conditions may be caused by or related to other health conditions that are not effectively treated by typical orthopaedic regimens. For example, patients presenting symptoms of a musculoskeletal condition may have an underlying health condition not directly indicated by the symptoms that may be a primary contributing factor to the symptoms. As a specific example, a patient with poor sleep hygiene who is a prolonged heavy smoker and is experiencing a mood disorder may present with unspecified back pain. Conventional treatment methods may dictate surgery, physical therapy, or prescription of a pain medication, none of which address the other factors that are the likely culprits of the back pain. Prolonged smoking is also bad for the health of the discs in the patient's neck and back and can cause the disc to degenerate at a more rapid rate, leading to worse back pain. In addition, data has shown that patients who have accompanying behavioral health concerns are more likely to have trouble with coping skills and their perception of pain and that including psychotherapy in their care can significantly reduce the need for pharmacotherapy.

Patients who are severely obese and get hip or knee replacements can wear out the artificial joints faster as a result of their morbid obesity, therefore needing a revision surgery which is a technically more challenging procedure, with higher complication rates and lower predictability of outcome. Meanwhile, a weight loss program as part of their treatment plan can help them lose weight, thus relieving a lot of the pressure on their joint, making the pain insignificant enough that the patient doesn't even need a first joint replacement, much less a second.

Conventional treatment methods have also contributed to the recent and ongoing epidemic of opiate use disorder (OUD). Without addressing the other contributing factors to the musculoskeletal condition, the patient continues to experience pain and rely on opiates, increasing dependency and, in some tragic cases, leading to overuse and/or death.

Patients with inflammatory arthritis/autoimmune issues, who often complain of pain in multiple musculoskeletal areas, are also often missed because they are not appropriately treated and the necessary laboratory data to help with the diagnosis is not often done. This leads to patients sometimes having multiple different surgeries (and continuing to have significant pain) for conditions, several of which can now be treated with medications such as disease-modifying antirheumatic drugs (DMARDs) if the appropriate laboratory testing is done.

Uncontrolled diabetes has been very well-documented to wreak havoc on the nerves and can cause neuropathy (pain in the hands and feet). This diagnosis is often missed with conventional treatment, as appropriate laboratory testing is not typically done. When diagnosed and treated appropriately, the blood glucose of someone with diabetes can return to a normal level and prevent worsening of such symptoms. Also, when patients have poorly controlled diabetes, they are at a higher risk for an infection after surgery.

Implementations of the value-based method of treating a musculoskeletal condition described below may address some or all of the problems described above. A particular benefit of the method is that it may be implemented by surgeons and non-surgeons alike, as the relative scarcity of orthopaedic surgeons makes access to them very difficult for many Americans. The method excludes resorting to surgery as a primary approach and instead focuses on a whole-health approach to identify and intervene in a host of health conditions that may contribute to the patient's pain. The inventor of the method has discovered that, by using the method to treat musculoskeletal conditions, fewer than one percent of patients are referred for surgery, and greater than ninety-eight percent of patients that complete the protocols associated with the method experience complete or significant pain relief and dramatically improved function, including being able to return to work, resume their activities of daily living. Review of the prescription drug monitoring program shows that they do all of these while not seeking opiates from any other physician in the state.

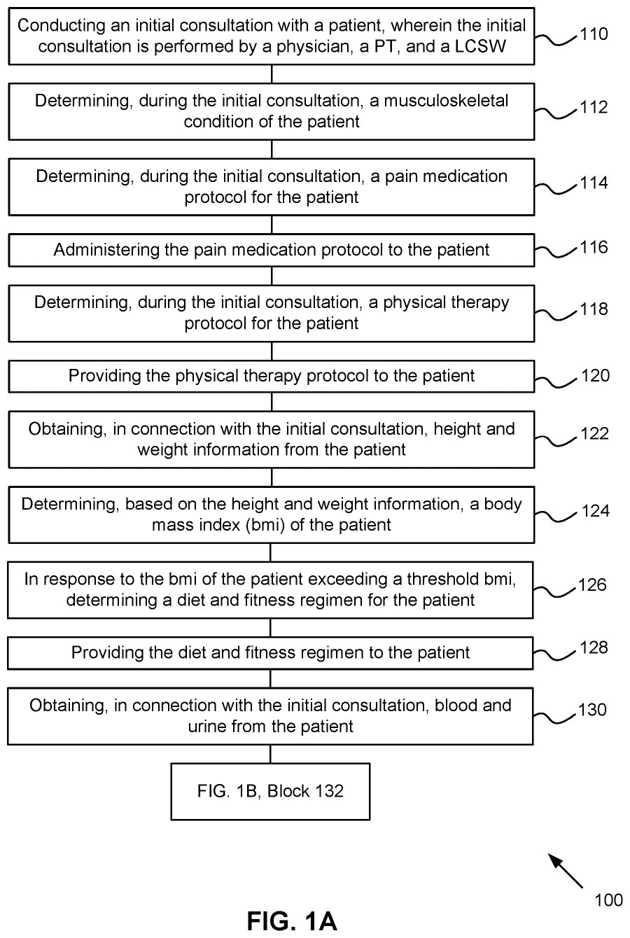

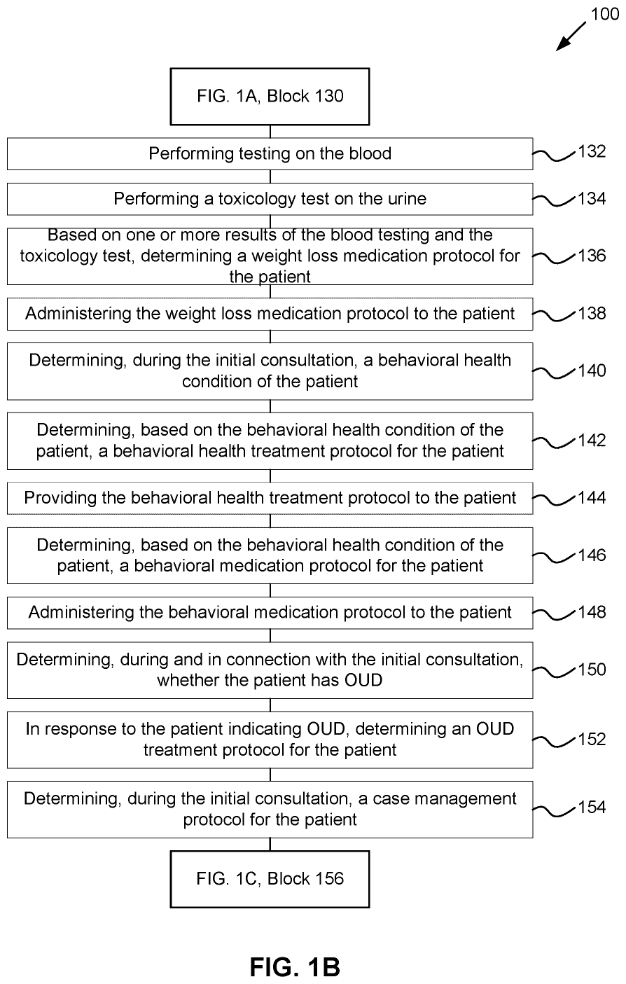

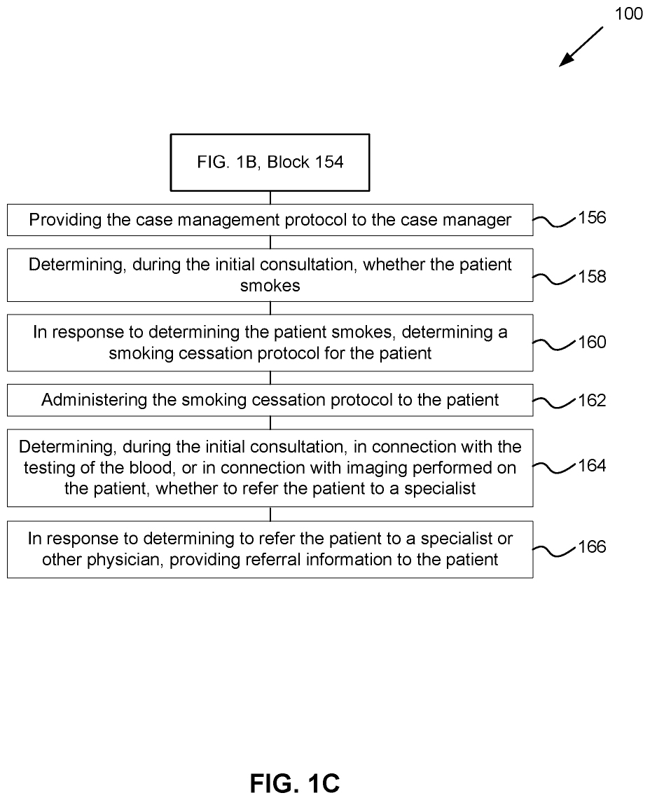

FIGS. 1A-C illustrate a method 100 of treating a musculoskeletal condition, according to an implementation. The method 100 may include conducting an initial consultation with a patient (block 110). The initial consultation may be performed by a physician, a physical therapist (PT), and a licensed clinical social worker (LCSW). In various implementations, the initial consultation may further include a segment wherein the LCSW interviews the patient alone. This may help the patient feel more comfortable with the treatment process and address concerns about their health in a private setting. Patient buy-in may be an important factor in successful treatment, and having a compassionate and understanding interaction during the initial consultation may help build trust with the patient. During or in connection with the initial consultation, non-medical information may be collected about the patient, with may include asking the patient questions about the patient's housing security, food security, health insurance, employment, and disability compensation.

The method 100 may include determining, during the initial consultation, the musculoskeletal condition of the patient (block 112).

The method 100 may include determining, during the initial consultation, a pain medication protocol for the patient (block 114). The pain medication protocol may include prescribing to the patient a pain reliever such as an analgesic. The analgesic may include one or more of methylprednisolone, lidocaine, amitriptyline, meloxicam, acetaminophen, cyclobenzaprine, and a nonsteroidal anti-inflammatory drug (NSAID). According to various implementations of the method 100, the pain medication protocol includes 7 different classes of pain medication and does not include any opiates. The pain medication protocol may be based on the patient indicating a desire for a pain medication. In response to the patient not desiring a pain medication, the pain medication protocol may include recommending a type and/or dosage of an over-the-counter medication and/or an herbal remedy. In response to the patient not desiring a pain medication, the pain medication protocol may include no pain medication.

In some implementations, such as when the patient does not indicate diabetes, the pain medication protocol may include prescribing and/or administering 4 mg of methylprednisolone once per day with food or drink for six days. In response to the patient indicating significant pain relief that plateaus after taking 4 mg of methylprednisolone once per day for six days, the pain medication protocol may further include six more days of 4 mg of methylprednisolone once per day with food or drink.

In some implementations, the pain medication protocol may include application of 2 or 3 patches of 4% or 5% lidocaine for 12 hours per day for sixty days.

The patient may indicate sleep problems, anxiety, and/or depression. In such implementations, the pain medication protocol may include prescribing and/or administering at least 10 mg and no greater than 75 mg of amitriptyline once per day before bed for sixty days.

In various implementations, such as when the patient does not indicate OUD, the pain medication protocol may include prescribing and/or administering 300 mg of gabapentin per day before bed for sixty days. The dosage may be titrated up to no more than 1500 mg gabapentin per day.

The pain medication protocol may, in various implementations, include prescription and/or administration of 7.5 mg of meloxicam once per day or twice per day with food or drink for sixty days.

In some implementations, the pain medication protocol may include administration of 500 mg of acetaminophen once every six hours. The pain medication protocol may include administration of 325 mg of acetaminophen once every four hours.

The pain medication protocol may include prescription and/or administration of at least 5 mg of cyclobenzaprine once per day. In some implementations, the patient may indicate prior use of cyclobenzaprine. The pain medication protocol may, in such implementations, include prescription and/or administration of 10 mg of cyclobenzaprine once per day.

In some implementations, the pain medication protocol may include prescription and/or administration of an NSAID. The pain medication protocol may further include prescription and/or administration of 20 mg of omeprazole once or twice per day for as long as the NSAID is taken.

The method 100 may include administering the pain medication protocol to the patient (block 116). Administering the pain medication protocol may include providing the patient with a prescription for a pain medication and instructions for using the pain medication.

Administering the pain medication protocol may include directly providing the pain medication indicated in the pain medication protocol to the patient with instructions for using the pain medication. Administering the pain medication protocol may include directly administering the pain medication to the patient. For example, the pain medication may be directly administered by providing the patient with a single oral dose which is immediately taken by the patient, or by injecting the patient with the pain medication. Administering the pain medication may include providing the patient with a recommendation of one or more over-the-counter pain medications, a dosage recommendation, and/or one or more instructions for taking the pain medication.

As used herein, the root term “administer” may refer to applying one or more steps or instructions associated with a protocol. For example, “administering a protocol” may refer to performing the steps of the protocol according to one or more instructions associated with the steps or the protocol. As another example, “administering a medication” may refer to providing a patient with a medication directly, such as by injecting the patient or providing a single oral dose that is immediately taken by the patient, or indirectly, such as by recommendation or prescription of a medication.

The method 100 may include determining, during the initial consultation, a physical therapy protocol for the patient (block 118). The physical therapy protocol may include one or more steps or instructions associated with one or more exercises for the patient. The physical therapy protocol may include instructions on the duration and/or frequency of the exercises. The physical therapy protocol may include instructions may include a period over which the physical therapy protocol is to be followed. As a specific example, the physical therapy protocol may include performance of one or more exercises two times a week for six weeks. The physical therapy protocol may include instructing the patient on one or more exercises, helping the patient perform the exercises, demonstrating the exercises, monitoring performance of the exercises, and so forth. The exercises may include one or more of a range of motion technique, a stretching technique, a strengthening technique, a circulation improvement technique, and a fluid mobilization technique.

The physical therapy protocol may include a duration for implementing the protocol. For example, the duration may be two weeks, four weeks, six weeks, a month, three months, and so forth. At its conclusion, the physical therapy protocol may include an appointment with the PT and the patient to assess the patient's progress in areas such as strength, flexibility, range of motion, and pain level. Based on the patient's condition at the appointment, an updated physical therapy protocol may be determined. For example, the physical therapy protocol may include six weeks of in-office physical therapy. The patient's condition at the end of the six weeks may indicate updating the protocol to include one or more home exercises performed most days of the week indefinitely.

The physical therapy protocol may include re-evaluation of the patient at a conclusion of the physical therapy protocol. For example, the patient may meet with the PT to discuss whether the physical therapy protocol was successful. As another example, the patient may provide a report on whether the physical therapy protocol was successful. The report may be a narrative report. The report may be a question-and-answer report. The report may be a multiple-choice report where the patient selects the most accurate answer to a given question.

The method 100 may include administering the physical therapy protocol to the patient (block 120). The physical therapy protocol may be administered in-person, such as during appointments with the PT and the patient. The physical therapy protocol may be administered remotely, such as during video conference appointments with the PT and the patient. The physical therapy protocol may be administered virtually, such as by providing the patient with pre-recorded videos of the exercises associated with the physical therapy protocol. The physical therapy protocol may be administered in writing, such as through prescription of the exercises with instructions on how to perform the exercises.

The method 100 may include obtaining, in connection with the initial consultation, height and/or weight information about the patient (block 122). The height and/or weight information may be obtained from the patient. For example, the height and/or weight information may be self-reported by the patient. As another example, the height and/or weight of the patient may be measured during, directly before, or directly after the initial consultation. The height and/or weight information may be obtained from a third party, such as from a medical chart obtained from the patient's primary care provider (PCP).

The method 100 may include determining, based on the height and weight information, a body mass index (BMI) of the patient (block 124). The BMI of the patient may be used to develop one or more protocols for treating the patient's musculoskeletal condition. For example, the BMI of the patient may be used to determine whether the patient is overweight, underweight, or within a healthy weight range.

In response to the BMI of the patient exceeding a threshold BMI, the method 100 may include determining a diet and fitness protocol for the patient (block 126). Alternatively, the BMI of the patient may be below a lower threshold for a healthy BMI. The diet and fitness protocol may be based on whether the patient's BMI is above or below a health range for BMI. The method 100 may include administering the diet and fitness protocol to the patient (block 128). In some implementations, the initial consultation may be additionally performed by a registered dietician, either simultaneously with the other practitioners, or during a different segment of the initial consultation from the other practitioners. The diet and fitness protocol may be determined and/or administered by the registered dietician.

The method 100 may include obtaining, in connection with the initial consultation, blood and urine from the patient (block 130). The method 100 may include performing testing on the blood (block 132). The testing may include one or more of a metabolic panel, a lipid panel, a thyroid panel, a diabetes panel, a nutrition panel, and a vitamin panel. The method 100 may include performing a toxicology test on the urine to determine whether the patient has engaged in substance abuse (block 134).

In various implementations, the lipid panel may include measurement of one or more of the patient's low-density lipoprotein (LDL) cholesterol, high-density lipoprotein (HDL) cholesterol, total cholesterol, and triglycerides. The diabetes panel may include measurement of one or more of the patient's hemoglobin A1e, fasting glucose plasma, and non-fasting glucose. The nutrition panel may include measurement of one or more of the patient's retinol, blood urea nitrogen (BUN), serum albumin, prealbumin, creatinine, and complete blood count (CBC). The vitamin panel may include measurement of one or more of the patient's vitamin A, vitamin E, vitamin D, and vitamin K1 levels.

The method 100 may include determining a weight loss medication protocol for the patient based on one or more results of the blood testing and the toxicology test (block 136). Determining the weight loss medication protocol may be based on one or more other factors, such as substance abuse history, indications for diabetes, and so forth. Particular medications may include one or more of phentermine, orlistat, and semaglutide. The method 100 may include administering the weight loss medication protocol to the patient (block 138).

In some implementations, the initial consultation may additionally be performed by a nurse practitioner (NP), either simultaneously with the other practitioners or during a different segment of the initial consultation. The NP may determine and/or administer one or more of the protocols described herein, including the pain medication protocol and the weight loss medication protocol, and, as described below, the behavioral medication protocol and the smoking cessation protocol.

In some implementations of the method 100, the diet and fitness protocol may include a first phase and a second phase. The first phase may be determined during or in connection with the initial consultation. The first phase may start immediately after the initial consultation. The second phase may be determined at a follow-up appointment at a conclusion of the first phase. For example, the first phase may have a duration of one week, and the second phase may be determined at the end of the week. The diet and fitness protocol, and/or the weight loss medication protocol, may be updated for the second phase based on the patient's results during the first phase.

The first phase may include calorie restriction to 2,000 kcal per day. The patient may be restricted from consuming juice and/or soda, though diet soda may be permitted. The first phase may include intermittent fasting, such as where no calories are consumed between 8 pm and 8 am. In general, the intermittent fasting may have a daily cycle where the patient is permitted to consume calories only during a certain period, such as a period of two hours, three hours, four hours, or six hours. It may be easiest for the patient to avoid consuming calories during periods when they are primarily sleeping or engaging in other activities where they cannot eat. The first phase may include thirty minutes of cardiovascular exercise three times a week. The first phase may include maintaining a food, activity, and/or sleep diary for at least three days. The three days may include two active days, e.g., workdays, and one leisure day, e.g., a day the patient has off work. The first phase may additionally include weight measurement avoidance by the patient. Because of the relatively short duration of the first phase and the intensity of the first phase activities and requirements, weight measurement avoidance may prevent the patient from becoming discouraged, such as by a perceived disparity between the patient's weight loss (or gain) and the effort expended by the patient.

The diet and fitness protocol for an underweight patient may be similar to that of an overweight patient. The protocol for an underweight patient may include a diet of between 2000 kcal and 4,000 kcal. The protocol for an underweight patient may include diet supplementation with one or more protein shakes, or otherwise increased protein intake, in conjunction with a high calorie diet.

The second phase may be based on the results of the first phase. In some implementations, the requirements and activities of the first phase may be continued into the second phase, such as for patients that struggled with the first phase or were not able to strictly comply with the requirements of the first phase. In some implementations, such as when a patient successfully completed the first phase, the activities and/or restrictions may be updated. The second phase may include calorie restriction to 1,500 kcal per day. The second phase may include nutrient supplementation, which may be based on one or more results of the blood testing. The second phase may include counseling on one or more of sleep hygiene, diet, or exercise, which counseling may be based on the food, activity, and/or sleep diary recorded during the first phase.

In some implementations, the weight loss medication protocol may be determined during the second phase. For patients that do not indicate a history of substance abuse or OUD, the weight loss medication protocol may include administration of phentermine. For patients with a BMI of 30 or greater, the weight loss medication protocol may include prescription and/or administration of 37.5 mg of phentermine once per day. For patients with a BMI of at least twenty-seven and less than thirty, the weight loss medication protocol may include prescription and/or administration of either 15 mg of phentermine once per day or 18.75 mg of phentermine once per day. For example, a patient indicating one or more of diabetes, high cholesterol, and high blood pressure may be given either 15 mg of phentermine once per day or 18.75 mg of phentermine once per day. A patient that indicates recent and/or rapid weight gain may be given 15 mg of phentermine once per day. A patient that indicates chronically high BMI or chronic weight gain may be given 18.75 mg once per day. In implementations where the patient indicates no or minimally reduced appetite after a period of time at 15 mg or 18.75 mg per day, the weight loss medication protocol may include increasing the dosage of phentermine to 37.5 mg of phentermine once per day.

In some implementations, phentermine may be contraindicated for the patient, such as where the patient has a history of substance abuse. The weight loss medication protocol may include prescription and/or administration of a different weight loss medication, such as orlistat. Such a protocol may include administration of 120 mg of orlistat three times a day before meals. Some patients may experience intolerable diarrhea on orlistat. In such cases, the weight loss medication protocol may include reducing the administration of orlistat to 120 mg twice per day or once per day before meals.

In some implementations, the patient may indicate diabetes, such as by having a high Ale or high fasting blood glucose level. In such cases, the weight loss medication protocol may include prescription and/or administration of 0.25 mg of semaglutide once per week for two weeks then 0.5 mg of semaglutide once per month thereafter. The same protocol may also be implemented for patients without diabetes. For patients with diabetes or pre-diabetes, the weight loss medication protocol may include daily blood sugar measurement.

The method 100 may include determining, during the initial consultation, a behavioral health condition of the patient (block 140). For example, the method 100 may include determining whether the patient indicates one or more of an anxiety disorder, a depressive disorder, and a personality disorder. Determining the behavioral health condition of the patient may include determining whether the patient indicates anxiety, chronic physical health problems, frequent primary care visits, or seeking of reassurance regarding the patient's health.

The method 100 may include determining, based on the behavioral health condition of the patient, a behavioral health treatment protocol for the patient (block 142). The behavioral health treatment protocol may be based on a particular condition indicated for the patient. The behavioral health treatment protocol may include one or more of non-facilitated self-help, guided self-help, counseling, cognitive behavioral therapy (CBT), behavioral activation (BA), physical exercise, mindfulness, meditation, interpersonal psychotherapy (IPT), and short-term psychodynamic psychotherapy (STPP). The method 100 may include administering the behavioral health treatment protocol to the patient (block 144).

The method 100 may include determining, based on the behavioral health condition of the patient, a behavioral medication protocol for the patient (block 146). In various implementations, the behavioral health treatment protocol and the behavioral medication protocol may be interdependent. The behavioral medication protocol may include prescription or recommendation of one or more medications, such as a selective serotonin reuptake inhibitor (SSRI), a serotonin and norepinephrine reuptake inhibitor (SNRI), an anticonvulsant, an anxiolytic, a tricyclic antidepressant (TCA), a tetracyclic antidepressant (TeCA), a monoamine oxidase inhibitor (MAOI), a betablocker, an antihistamine, an antipsychotic, and/or an herbal compound. In various implementations, the behavioral health treatment protocol does not include benzodiazepine or an antipsychotic. When pharmacological intervention is indicated, the method 100 may include determining an age of the patient and, in response to the patient being younger than thirty years old, monitoring the patient for suicidal thoughts or self-harm, as various behavioral medications may induce or increase such thoughts or behavior. The method 100 may include administering the behavioral medication protocol to the patient (block 148).

In various implementations, determining the behavioral health condition of the patient may include assessing the patient for generalized anxiety disorder (GAD). Assessing the patient for GAD may include assessing one or more of a distress level of the patient, a functional impairment of the patient, physical symptoms of the patient, a medical history of the patient, whether the patient indicates a comorbid condition, whether the patient has a history of substance abuse or OUD, whether the patient has been previously diagnosed with GAD, and whether the patient responds or has previously responded to treatment for GAD. Assessing the patient for GAD may further include determining one or more of a duration of symptoms, a distress level of the patient, impairment of functioning of the patient, a past history of anxiety of the patient, and whether the patient currently has, or previously had another mood disorder. The patient may be determined to have GAD based on assessment of the patient indicating one or more of excessive worry, difficulty controlling the worry, restlessness, fatigue, difficulty concentrating, irritability, muscle tension, and disturbed sleep.

Treatment for GAD may include therapy, medication, or a combination of both. Treatment may be stepped, and may include initially low-level intervention, moderate-level intervention when the patient does not respond to low-level intervention, and heightened intervention when the patient exhibits heightened symptoms or does not respond to lower levels of intervention.

Upon determining the patient indicates GAD, the behavioral health treatment protocol may include communicating a GAD diagnosis to the patient. The behavioral health treatment protocol may include treatment of comorbid conditions in GAD such as with another anxiety disorder or depressive disorder. In implementations where the patient indicates substance abuse and/or OUD, both of which may contribute to GAD, the behavioral health treatment protocol may include substance abuse treatment and/or OUD treatment. The behavioral health treatment protocol may include education on GAD, such as contributing and/or exacerbating factors, treatment and management options, and so forth. The behavioral health treatment protocol may include recommendations for an over-the-counter medication and/or an herbal treatment. In cases where the patient indicates low-level GAD, the behavioral health treatment protocol may include non-facilitated self-help. Low-level GAD may be determined in response to the patient indicating one or more of few or intermittent GAD symptoms, subclinical presentation of GAD symptoms, mild distress with no or limited functional impairment, no comorbid anxiety or mood disorders, no past history of anxiety or mood disorders, and disinterest in active treatment for GAD. In cases where the patient indicates moderate GAD, the behavioral health treatment protocol may include one or more of the guided self-help, counseling, and the CBT. Moderate GAD may be indicated in response to the patient indicating one or more of meeting diagnostic criteria for GAD, clinically significant distress or impairment, a comorbid anxiety or mood disorder, and interest in active treatment for GAD.

In cases where the patient indicates low-level GAD, the behavioral health treatment protocol may include non-facilitated self-help. Low-level GAD may be determined in response to the patient indicating one or more of few or intermittent GAD symptoms, subclinical presentation of GAD symptoms, mild distress with no or limited functional impairment, no comorbid anxiety or mood disorders, no past history of anxiety or mood disorders, and disinterest in active treatment for GAD. In cases where the patient indicates moderate GAD, the behavioral health treatment protocol may include one or more of the guided self-help, counseling, and the CBT. Moderate GAD may be indicated in response to the patient indicating one or more of meeting diagnostic criteria for GAD, clinically significant distress or impairment, a comorbid anxiety or mood disorder, and interest in active treatment for GAD.

In some cases, the behavioral health condition of the patient may be treated without medication. In such cases, the behavioral medication protocol may include not prescribing and/or administering a behavioral medication to the patient. In some cases, the patient may indicate one or more of marked functional impairment, clinically significant distress or impairment with inadequate response to non-pharmacological treatment of GAD, and/or a past history of an anxiety or mood disorder. In such cases, the behavioral medication protocol may include pharmacological intervention.

In some implementations, treatment for GAD may be stepped. The behavioral health treatment protocol may initially include low-level treatment. After a period of time, or based on symptoms exhibited by the patient, it may be determined the patient is not responding to the low-level treatment. The behavioral health treatment protocol may include re-evaluation and updating of the protocol to moderate-level treatment. As a specific example, the method 100 may include determining the patient indicates unimproved GAD after implementation of the behavioral health treatment protocol. An updated treatment protocol may be determined, which may include one or more of non-facilitated self-help, guided self-help, and participation in a psychoeducational group. The updated treatment protocol may be administered to the patient.

In some cases, it may be determined the patient still exhibits unimproved GAD after implementation of moderate-level treatment. A heightened treatment protocol may be determined for the patient, which may include evaluating one or more of a behavioral health history of the patient, a prior treatment experience of the patient, and a preference of the patient. The behavioral health treatment protocol may be further updated and/or heightened to include one or more of the CBT, applied relaxation, and pharmacological intervention or increased pharmacological intervention. For example, the behavioral medication protocol may be determined in response to the patient still indicating unimproved GAD after implementation of one or more previous behavioral health treatment protocols. The heightened treatment protocol may be administered to the patient. In some cases, the patient may exhibit functional impairment during the initial consultation, and the heightened treatment protocol may be administered immediately, instead of stepping the patient through lower levels of treatment.

In various implementations, non-facilitated self-help may include utilization of CBT principles by the patient with minimal therapist contact. The behavioral health treatment protocol may include providing self-guided materials on CBT to the patient. The protocol may include having the patient do the non-facilitated self-help for at least six weeks, and thereafter re-evaluating the patient's behavioral health condition.

In various implementations, the behavioral health treatment protocol may include guided self-help for the patient. Guided self-help may include one or more of utilization by the patient of CBT principles for at least six weeks. Guided self-help may include counseling with the patient and a practitioner trained in CBT. The counseling sessions may occur for twenty to thirty minutes at least once every four weeks. The counseling session may occur bi-weekly, bi-monthly, weekly, multiple days a week, or daily. In some implementations the counseling session may occur no more than once a day.

In various implementations, the behavioral health treatment may include group treatment and/or activities. For example, the protocol may include participation by the patient in a psychoeducational group, where a therapist-to-participant ratio is no greater than one to twelve. The psychoeducational group may occur in in two-hour sessions weekly for at least six weeks. Patients may be provided with self-help manuals and may observe presentations by a trained practitioner. Participation by the patients may be interactive, observational, or both.

The behavioral medication protocol for GAD may include prescription of an anticonvulsant, an SSRI, an SNRI, a TCA, a TeCA, an anxiolytic, an antihistamine, and/or an herbal compound. The anticonvulsant may, for example, include pregabalin. The SSRI may include at least one of sertraline, paroxetine, fluoxetine, citalopram, and escitalopram. The antipsychotic may include quetiapine. The TCA may include at least one of clomipramine and imipramine. The TeCA may include mirtazapine. The anxiolytic may include buspirone. The antihistamine may include hydroxyzine. The herbal compound may include at least one of chamomile, gingko biloba, and lavender oil. In heightened cases of GAD, the behavioral medication protocol may have a duration of at least one year. In response to the patient indicating a behavioral health crisis, the behavioral medication protocol may include administering benzodiazepine to the patient until the patient no longer indicates the behavioral health crisis.

Examples of the behavioral medication protocol for GAD may include: administering to the patient at least 150 mg and no more than 600 mg of pregabalin per day in divided doses; administering to the patient at least 40 mg of the betablocker per day, titrated up to no more than 120 mg per day; administering to the patient at least 15 mg and no more than 60 mg of buspirone per day in divided doses; administering to the patient at least 50 mg and no more than 100 mg of hydroxyzine per day in divided doses; administering to the patient at least 50 mg and no more than 300 mg of quetiapine per day; administering to the patient at least 50 mg and no more than 250 mg of clomipramine per day, or, for a patient less than 25 years old, initially administering to the patient 10 mg of clomipramine per day; administering to the patient at least 75 mg and no more than 200 mg of imipramine per day in divided doses, or, for a patient less than 18 years old, initially administering to the patient no more than 25 mg of imipramine every four days, which may be increased over time in 50 mg increments; administering to the patient at least 15 mg and no more than 30 mg of mirtazapine per day before bed; administering to the patient at least 220 mg and no more than 1,500 mg of chamomile per day; administering to the patient at least 240 mg and no more than 480 mg of gingko biloba per day; administering to the patient at least 80 mg and no more than 160 mg of lavender oil per day.

In some implementations, the patient may indicate depression when the behavioral health condition of the patient is determined. In cases where the patient exhibits mild depression, the patient may be provided with materials for one or more of structured CBT, structured BA, problem-solving, or psychoeducation. The behavioral health treatment protocol may include at least six therapy sessions with the patient and a trained practitioner. The behavioral health treatment protocol includes at least eight sessions of individual CBT with a practitioner trained in CBT. The behavioral health treatment protocol may include at least eight sessions of individual BA with a practitioner in BA. The behavioral health treatment protocol may include at least 8 sessions of IPT with a practitioner trained in IPT. The behavioral health treatment protocol may include at least 8 sessions of IPT with a practitioner trained in IPT. The behavioral health treatment protocol may include at least 8 counseling sessions with a practitioner trained in counseling on depression. The behavioral health treatment protocol may include at least eight sessions of STPP with a practitioner trained in STPP.

In some implementations, the behavioral health treatment protocol for mild depression may include one or more different forms of group therapy. The treatment protocol may include at least eight sessions of group CBT guided by at least two practitioners trained in CBT. The behavioral health treatment protocol may include at least eight sessions of group BA guided by at least two practitioners trained in BA. The behavioral health treatment protocol may include at least ten weeks of group physical exercise guided by a practitioner trained in physical activity intervention for depression. The behavioral health treatment protocol may include at least eight weeks of group mindfulness guided by at least two practitioners trained in mindfulness. The behavioral health treatment protocol may include at least eight weeks of group meditation guided by at least two practitioners trained in meditation.

In some implementations, the patient may indicate moderate to severe depression when the behavioral health condition of the patient is determined. The behavioral health treatment protocol may include at least sixteen sessions of individual CBT with a practitioner trained in CBT. The behavioral health treatment protocol may include at least twelve sessions of individual BA with a practitioner trained in BA. The behavioral health treatment protocol may include at least six sessions of individual problem-solving training with a practitioner trained in counseling on problem-solving. The behavioral health treatment protocol includes at least 12 counseling sessions with a practitioner trained in counseling on depression. The behavioral health treatment protocol may include at least sixteen sessions of STPP with a practitioner trained in STPP. The behavioral health treatment protocol includes at least sixteen sessions of IPT with a practitioner trained in IPT. The behavioral health treatment protocol may include at least six therapy sessions with a practitioner trained in counseling on depression and may further include providing the patient with materials for one or more of structured CBT, structured BA, problem-solving, or psychoeducation.

In some implementations, moderate to severe depression may be treated in a group setting, including those group therapy protocols described above regarding GAD. For example, the behavioral health treatment protocol may include at least ten weeks of group physical exercise guided by a practitioner trained in physical activity intervention for depression. Other examples may include those described above regarding mild depression with longer duration.

In some cases of depression, pharmacological intervention may not be indicated. In such cases, the behavioral medication protocol may include not administering medication. In some cases of depression, pharmacological intervention may be indicated, such as in cases where a patient expresses interest in medication or when the patient's depression does not improve with therapy alone. In such implementations, the behavioral medication protocol may include prescribing and/or administering a medication such as an antidepressant. For example, the behavioral medication protocol may include the patient taking the SSRI for at least six months.

Examples of the behavioral medication protocol for patients exhibiting depression may include: administering to the patient at least 50 mg and no more than 200 mg of sertraline once per day, and increasing the dosage of the sertraline weekly by 50 mg; administering to the patient at least 20 mg and no more than 60 mg of fluoxetine daily in a single or a divided dose; administering to the patient at least 20 mg and no more than 50 mg of paroxetine once daily approximately when the patient rises from bed; administering to the patient at least 20 mg and no more than 40 mg of citalopram once daily, and increasing a dosage of the citalopram in three-or four-week intervals; administering to the patient at least 5 mg and no more than 20 mg of escitalopram once daily; and administering to the patient at least 15 mg and no more than 30 mg of mirtazapine once daily before bed or in two divided doses, and increasing the dosage of mirtazapine after at least two weeks and no more than four weeks from the initial administering of mirtazapine up to no more than 45 mg.

The method 100 may include determining, during and/or in connection with the initial consultation, whether the patient has opiate use disorder (OUD) (block 150). The method 100 may include, in response to the patient indicating OUD, determining an OUD treatment protocol for the patient (block 152). In implementations where the patient does not indicate OUD, the OUD treatment protocol may include recommending no OUD treatment. In implementations where OUD is indicated, the OUD treatment protocol may include prescribing and/or recommending a combination of buprenorphine and naloxone. The OUD treatment protocol may include a two-week prescription for the medications and requirement that the patient attend a follow-up appointment and provide a urine sample so that a follow-up toxicology test can be performed. Subsequent phases of the OUD treatment protocol may be based on the results of the follow-up toxicology test.

The method 100 may include determining, during the initial consultation, a case management protocol for the patient (block 154). The case management protocol may include monitoring by a case manager of the patient's social determinants of health. The case management protocol may include monitoring reports for the patient from a prescription drug monitoring program. The case management protocol may include a duration. For example, the case management protocol may be executed for at least three months after the patient stops attending all appointments associated with treating the musculoskeletal condition. For OUD patients, the duration may be no more than six months. The method 100 may include providing the case management protocol to the case manager (block 156).

The method 100 may include determining, during the initial consultation, whether the patient smokes (block 158). The method 100 may include, in response to determining the patient smokes, determining a smoking cessation protocol for the patient (block 160). In some implementations, the patient may not smoke, and the smoking cessation protocol may indicate no intervention is necessary. The smoking cessation protocol may include prescribing, providing, and/or administering one or more smoking cessation medications, such as a nicotine patch, varenicline, and/or bupropion. The method 100 may include administering the smoking cessation protocol to the patient (block 162).

The smoking cessation protocol is based on an average number of cigarettes smoked by the patient per day. For example, for a patient that smokes approximately 10 cigarettes per day, the smoking cessation protocol may include administering to the patient a 21 mg nicotine patch for 24 hours a day. For a patient that smokes approximately 30 cigarettes per day, the smoking cessation protocol may include administering to the patient a 21 mg nicotine patch and a 14 mg nicotine patch for 24 hours a day. The smoking cessation protocol further may include lowering a dosage of the nicotine patch over a period in a range from eight weeks to twelve weeks. The patient is completely weaned off the nicotine patch at the end of the period.

The smoking cessation protocol may include administering to the patient 0.5 mg of varenicline once a day for three days, then 0.5 mg of the varenicline twice a day for four days, and then 1 mg of the varenicline twice a day for 11 weeks. An initial dose of the varenicline may be administered approximately one week before a date on which the patient projects to stop smoking. The initial dose of the varenicline may be administered to the patient at least eight days and no more than thirty-five days before a date on which the patient projects to stop smoking. The smoking cessation protocol may include administering to the patient 150 mg of bupropion once a day for three days, then 150 mg of the bupropion twice a day for approximately twelve weeks. The initial dose of bupropion may be administered at least one week and no more than two weeks before a date on which the patient projects to stop smoking.

The method 100 may include determining, during the initial consultation, in connection with the testing of the blood, or in connection with imaging performed on the patient, whether to refer the patient to a specialist (block 164). Such determination may be based on one or more of vitals of the patient, abnormal lab results, the imaging, and the patient reporting one or more of unintentional weight loss, unexplained night sweats, active vomiting, active diarrhea, current fever, complaint by the patient of objective muscle weakness, acute problems with bowel or bladder function, dizziness, chest pain, and dyspnea. The method 100 may include, in response to determining to refer the patient to a specialist or other physician, providing referral information to the patient (block 166). For example, the patient may be determined to have an acute musculoskeletal injury requiring surgery and may be referred to an orthopedic surgeon. As another example, determining to refer the patient to a specialist or other physician may be in response to the patient indicating GAD and one or more of resistance to CBT, resistance to the behavioral medication protocol, severe functional impairment, persistent suicidal thoughts, multiple psychiatric comorbidities, and unimproved GAD after implementation of the heightened treatment protocol.

In some implementations, referral may not be indicated, and information may be provided to the patient indicating that referral is not necessary. Such information may further include an explanation of how treatment following a method such as method 100 without referral may improve the patient's health and provide relief from the musculoskeletal condition.

A feature illustrated in one of the figures may be the same as or similar to a feature illustrated in another of the figures. Similarly, a feature described in connection with one of the figures may be the same as or similar to a feature described in connection with another of the figures. The same or similar features may be noted by the same or similar reference characters unless expressly described otherwise. Additionally, the description of a particular figure may refer to a feature not shown in the particular figure. The feature may be illustrated in and/or further described in connection with another figure.

Elements of processes (i.e. methods) described herein may be executed in one or more ways such as by a human, by a processing device, by mechanisms operating automatically or under human control, and so forth. Additionally, although various elements of a process may be depicted in the figures in a particular order, the elements of the process may be performed in one or more different orders without departing from the substance and spirit of the disclosure herein.

The foregoing description sets forth numerous specific details such as examples of specific systems, components, methods and so forth, in order to provide a good understanding of several implementations. It will be apparent to one skilled in the art, however, that at least some implementations may be practiced without these specific details. In other instances, well-known components or methods are not described in detail or are presented in simple block diagram format in order to avoid unnecessarily obscuring the present implementations. Thus, the specific details set forth above are merely exemplary. Particular implementations may vary from these exemplary details and still be contemplated to be within the scope of the present implementations.

Related elements in the examples and/or embodiments described herein may be identical, similar, or dissimilar in different examples. For the sake of brevity and clarity, related elements may not be redundantly explained. Instead, the use of a same, similar, and/or related element names and/or reference characters may cue the reader that an element with a given name and/or associated reference character may be similar to another related element with the same, similar, and/or related element name and/or reference character in an example explained elsewhere herein. Elements specific to a given example may be described regarding that particular example. A person having ordinary skill in the art will understand that a given element need not be the same and/or similar to the specific portrayal of a related element in any given figure or example in order to share features of the related element.

It is to be understood that the foregoing description is intended to be illustrative and not restrictive. Many other implementations will be apparent to those of skill in the art upon reading and understanding the above description. The scope of the present implementations should, therefore, be determined with reference to the appended claims, along with the full scope of equivalents to which such claims are entitled.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements.

Where multiples of a particular element are shown in a FIG., and where it is clear that the element is duplicated throughout the FIG., only one label may be provided for the element, despite multiple instances of the element being present in the FIG. Accordingly, other instances in the FIG. of the element having identical or similar structure and/or function may not have been redundantly labeled. A person having ordinary skill in the art will recognize based on the disclosure herein redundant and/or duplicated elements of the same FIG. Despite this, redundant labeling may be included where helpful in clarifying the structure of the depicted examples.

The Applicant(s) reserves the right to submit claims directed to combinations and sub-combinations of the disclosed examples that are believed to be novel and non-obvious. Examples embodied in other combinations and sub-combinations of features, functions, elements and/or properties may be claimed through amendment of those claims or presentation of new claims in the present application or in a related application. Such amended or new claims, whether they are directed to the same example or a different example and whether they are different, broader, narrower or equal in scope to the original claims, are to be considered within the subject matter of the examples described herein. The invention has been described with reference to various specific and preferred embodiments and techniques. Nevertheless, it is understood that many variations and modifications may be made while remaining within the spirit and scope of the invention.

What is claimed is:

1. A method of treating a musculoskeletal condition, comprising:
conducting an initial consultation with a patient, wherein the initial consultation is performed by a physician, a physical therapist (PT), and a licensed clinical social worker (LCSW);
determining, during the initial consultation, the musculoskeletal condition of the patient;
determining, during the initial consultation, a pain medication protocol for the patient comprising one or more of methylprednisolone, lidocaine, amitriptyline, meloxicam, acetaminophen, cyclobenzaprine, and a nonsteroidal anti-inflammatory drug (NSAID), wherein the pain medication protocol does not comprise an opiate, quetiapine, benzodiazepine, or a stimulant, and wherein the pain medication protocol is based on the patient indicating a desire for a pain medication;
administering the pain medication protocol to the patient;
determining, during the initial consultation, a physical therapy protocol for the patient, wherein the physical therapy protocol comprises performance of one or more exercises two times a week for six weeks and re-evaluation of the patient at a conclusion of the physical therapy protocol;
administering the physical therapy protocol to the patient;
obtaining, in connection with the initial consultation, height and weight information from the patient;
determining, based on the height and weight information, a body mass index (BMI) of the patient;
in response to the BMI of the patient exceeding a threshold BMI, determining a diet and fitness protocol for the patient;
administering the diet and fitness protocol to the patient;
obtaining, in connection with the initial consultation, blood and urine from the patient;
performing testing on the blood, wherein the testing comprises one or more of a metabolic panel, a lipid panel, a thyroid panel, a diabetes panel, a nutrition panel, and a vitamin panel;
performing a toxicology test on the urine to determine whether the patient has engaged in substance abuse;
based on one or more results of the blood testing and the toxicology test, determining a weight loss medication protocol for the patient comprising one or more of phentermine, orlistat, and semaglutide;
administering the weight loss medication protocol to the patient;
determining, during the initial consultation, a behavioral health condition of the patient;
determining, based on the behavioral health condition of the patient, a behavioral health treatment protocol for the patient, wherein the behavioral health treatment protocol comprises one or more of non-facilitated self-help, guided self-help, counseling, cognitive behavioral therapy (CBT), behavioral activation (BA), physical exercise, mindfulness, meditation, interpersonal psychotherapy (IPT), and short-term psychodynamic psychotherapy (STPP);
administering the behavioral health treatment protocol to the patient; determining, based on the behavioral health condition of the patient, a behavioral medication protocol for the patient comprising one or more of a selective serotonin reuptake inhibitor (SSRI), a serotonin and norepinephrine reuptake inhibitor (SNRI), an anticonvulsant, an anxiolytic, a tricyclic antidepressant (TCA), a tetracyclic antidepressant (TeCA), a monoamine oxidase inhibitor (MAOI), a betablocker, an antihistamine, an antipsychotic, and an herbal compound, and wherein the behavioral health treatment protocol does not comprise benzodiazepine or an antipsychotic;
administering the behavioral medication protocol to the patient;
determining, during and in connection with the initial consultation, whether the patient has opiate use disorder (OUD);
in response to the patient indicating OUD, determining an OUD treatment protocol for the patient comprising buprenorphine and naloxone for two weeks, a follow-up appointment, and a follow-up toxicology test;
determining, during the initial consultation, a case management protocol for the patient comprising monthly monitoring by a case manager of the patient's social determinants of health and reports from a prescription drug monitoring program for the patient;
administering the case management protocol to the case manager; determining, during the initial consultation, whether the patient smokes;
in response to determining the patient smokes, determining a smoking cessation protocol for the patient comprising one or more of a nicotine patch, varenicline, and bupropion;
administering the smoking cessation protocol to the patient;
determining, during the initial consultation, in connection with the testing of the blood, or in connection with imaging performed on the patient, whether to refer the patient to a specialist based on one or more of vitals of the patient, abnormal lab results, the imaging, and the patient reporting one or more of unintentional weight loss, unexplained night sweats, active vomiting, active diarrhea, current fever, complaint by the patient of objective muscle weakness, acute problems with bowel or bladder function, dizziness, chest pain, and dyspnea; and
in response to determining to refer the patient to a specialist or other physician, providing referral information to the patient.

2. The method of claim 1, wherein the patient indicates GAD when the behavioral health condition of the patient is determined, and wherein the behavioral health treatment protocol further comprises one or more of communicating a GAD diagnosis to the patient, prioritized treatment of comorbid conditions in GAD with another anxiety disorder or depressive disorder, substance abuse treatment, OUD treatment, education on GAD, and an over-the-counter medication.

3. The method of claim 2, further comprising:
determining the patient indicates unimproved GAD after implementation of the behavioral health treatment protocol;
determining an updated treatment protocol comprising one or more of the non-facilitated self-help, the guided self-help, and participation in a psychoeducational group; and
providing the updated treatment protocol to the patient.

4. The method of claim 3, wherein the non-facilitated self-help comprises one or more of utilization of CBT principles for at least six weeks and minimal therapist contact.

5. The method of claim 3, wherein the guided self-help comprises one or more of utilization of CBT principles for at least six weeks and counseling for twenty to thirty minutes at least once every four weeks and no more than once per day.

6. The method of claim 3, wherein the psychoeducational group comprises a therapist-to-participant ratio of no greater than one to twelve in two-hour sessions for at least six weeks with a self-help manual and presentations by a trained practitioner, and wherein the participation is interactive, observational, or both.

7. The method of claim 3, further comprising:
determining the patient still indicates the unimproved GAD after implementation of the updated treatment protocol;
determining a heightened treatment protocol comprising one or more of the CBT, applied relaxation, and the behavioral medication protocol, wherein the behavioral medication protocol is determined in response to the patient still indicating unimproved GAD after implementation of the updated treatment protocol; and
providing the heightened treatment protocol to the patient.

8. The method of claim 7, wherein the behavioral medication protocol comprises the SSRI or the SNRI.

9. The method of claim 7, wherein, in response to the patient indicating a behavioral health crisis, the behavioral medication protocol comprises administering benzodiazepine until the patient no longer indicates the behavioral health crisis.

10. The method of claim 7, wherein determining the heightened treatment protocol comprises evaluating one or more of a behavioral health history of the patient, a prior treatment experience of the patient, and a preference of the patient.

11. The method of claim 7, wherein the heightened treatment protocol further comprises determining an age of the patient and, in response to the patient being younger than thirty years old, monitoring the patient for one or more of suicidal thinking and self-harm.

12. The method of claim 7, wherein the behavioral medication protocol has a duration of at least one year.

13. The method of claim 7, wherein determining to refer the patient to the specialist or other physician is in response to the patient still indicating unimproved GAD after implementation of the heightened treatment protocol.

14. The method of claim 2, further comprising:
determining the patient indicates functional impairment;
determining a heightened treatment protocol comprising one or more of the CBT, applied relaxation, and the behavioral medication protocol, wherein the behavioral medication protocol is determined in response to the patient the functional impairment; and
providing the heightened treatment protocol to the patient.

15. The method of claim 14, wherein the behavioral medication protocol comprises the SSRI or the SNRI.

16. The method of claim 14, wherein, in response to the patient indicating a behavioral health crisis, the behavioral medication protocol comprises administering benzodiazepine until the patient no longer indicates the behavioral health crisis.

17. The method of claim 14, wherein determining the heightened treatment protocol comprises evaluating one or more of a behavioral health history of the patient, a prior treatment experience of the patient, and a preference of the patient.

18. The method of claim 14, wherein the heightened treatment protocol further comprises determining an age of the patient and, in response to the patient being younger than thirty years old, monitoring the patient for one or more of suicidal thinking and self-harm.

19. The method of claim 14, wherein the behavioral medication protocol has a duration of at least one year.

20. The method of claim 14, wherein determining to refer the patient to the specialist or other physician is in response to the patient still indicating unimproved GAD after implementation of the heightened treatment protocol.
